(19) United States
(12) Patent Application Publication
(10) Pub. No.: US 2026/0250419 A1
(43) Pub. Date: Aug. 27, 2026

(54) RECOMBINANT PROTEIN COMPRISING ADDITIONAL DOMAIN

(71) Applicant: VACINO BIOTECH CO., LTD., TAIPEI (TW)

(72) Inventor: JIA-MING CHANG, TAIPEI (TW)

(21) Appl. No.: 19/407,469

(22) Filed: Dec. 3, 2025

Related U.S. Application Data
(60) Provisional application No. 63/727,342, filed on Dec. 3, 2024.

(51) Int. Cl. C07K 16/32; A61K 39/00; A61P 35/00

(52) U.S. Cl. CPC C07K 16/32; A61P 35/00; A61K 2039/505; C07K 2317/53; C07K 2317/92

(57) ABSTRACT

Disclosed herein is a recombinant protein comprising an additional domain; for example, a recombinant antibody comprising a transporter domain for penetrating the blood-brain barrier. A nucleic acid encoding the recombinant protein is also provided. Also disclosed herein is a method for treating or detecting a disease by use of the present recombinant protein.

### PRIORITY INFORMATION

The subject application claims priority to and benefit of U.S. Provisional Patent Application No. 63/727,342, filed Dec. 3, 2024, the content of which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the XML file containing the sequence listing is US16451.xml. The XML file is 48,975 bytes; was created on Dec. 2, 2025; and is being submitted electronically via Patent Center

### FIELD OF THE INVENTION

The present disclosure in general relates to the field of protein engineering. More particularly, the present disclosure relates to a recombinant protein comprising an additional domain.

### BACKGROUND OF THE INVENTION

Protein engineering is a rapidly advancing field that involves the deliberate modification of protein structures to enhance or introduce specific functional properties. By altering amino acid sequences, proteins can be tailored for improved stability, activity, specificity, or novel functionalities. These engineered proteins have broad applications across biotechnology, medicine, and industrial processes, enabling the development of more effective therapeutics, catalysts, and diagnostic tools.

A particularly promising approach within protein engineering is the addition of an auxiliary domain to a target protein. Incorporating an additional domain can confer new capabilities, such as enhanced binding affinity, altered substrate specificity, improved solubility, or regulatory control. This modular strategy allows for the creation of multifunctional proteins by combining distinct structural and functional elements, thereby expanding the utility and versatility of engineered proteins.

### SUMMARY OF THE INVENTION

As embodied and broadly described herein, a strategy for introducing an additional domain in a protein is provided by applying a pair sequences.

Accordingly, the present disclosure provides a recombinant protein comprising
a socket sequence comprising
   a pair of a socket upstream sequence selected from the group consisting of amino acid sequences of SEQ ID NOs: 1 to 5 and a socket downstream sequence selected from the group consisting of amino acid sequences of SEQ ID NOs: 6 to 10; or
   a pair of a socket upstream sequence selected from the group consisting of amino acid sequences of SEQ ID NOs: 11 to 15 and a socket downstream sequence selected from the group consisting of amino acid sequences of SEQ ID NOs: 16 to 20; and
a plug sequence directly linked to the socket upstream sequence and socket downstream sequence in the pair.

In some embodiments of the disclosure, the plug sequence forms a functional domain.

In some embodiments of the disclosure, the plug sequence comprises a size of no more than 50 kDa, 45 kDa, 40 kDa, 35 kDa, 30 kDa, 25 kDa, 20 kDa, 15 kDa, or 10 kDa. In another aspect, the plug sequence comprises a size of about 1 kDa to about 10 kDa, about 2 kDa to about 9 kDa, about 3 kDa to about 8 kDa, about 4 kDa to about 7 kDa, about 5 kDa to about 6 kDa, about 10 kDa to about 30 kDa, about 11 kDa to about 29 kDa, about 12 kDa to about 28 kDa, about 13 kDa to about 27 kDa, about 14 kDa to about 26 kDa, about 15 kDa to about 25 kDa, about 16 kDa to about 24 kDa, about 17 kDa to about 23 kDa, about 18 kDa to about 22 kDa, about 19 kDa to about 21 kDa, about 20 kDa.

In some embodiments of the disclosure, the plug sequence is selected from the group consisting of SEQ ID NOs. 21 to 49.

In some embodiments of the disclosure, the recombinant protein comprises the sequence selected from the group consisting of SEQ ID NOs. 50 to 54 or a substantially similar sequence having at least 85%, 86%, 87%, 88%, 89%, 90%, 91%, 92%, 93%, 94%, 95%, 96%, 97%, 98%, or 99% sequence identity.

In some embodiments of the disclosure, the recombinant protein further comprises a frame sequence. An example of the frame sequence is an antibody or a fragment thereof. An example of the antibody is trastuzumab.

In some embodiments of the disclosure, the socket sequence and plug sequence are integrated into the frame sequence or linked to a terminal of the frame sequence.

In some embodiments of the disclosure, the frame sequence is an antibody or a fragment thereof, and the socket sequence and plug sequence are integrated into a hinge region, linked to a terminal of a heavy chain or linked to a terminal of a light chain of the antibody or the fragment thereof.

The present disclosure also provides a recombinant antibody or a fragment thereof, comprising
a socket sequence comprising
   a pair of a socket upstream sequence selected from the group consisting of amino acid sequences of SEQ ID NOs: 1 to 5 and a socket downstream sequence selected from the group consisting of amino acid sequences of SEQ ID NOs: 6 to 10; or
   a pair of a socket upstream sequence selected from the group consisting of amino acid sequences of SEQ ID NOs: 11 to 15 and a socket downstream sequence selected from the group consisting of amino acid sequences of SEQ ID NOs: 16 to 20;
a plug sequence selected from the group consisting of SEQ ID NOs. 21 to 49 and directly linked to the socket upstream sequence and socket downstream sequence in the pair; and
a frame sequence of an antibody or a fragment thereof;
wherein the socket sequence and plug sequence are integrated into a hinge region, linked to a terminal of a heavy chain or linked to a terminal of a light chain of the antibody or the fragment thereof.

In some embodiments of the disclosure, the antibody is trastuzumab.

In some embodiments of the disclosure, the recombinant antibody or the fragment thereof comprises the sequence selected from the group consisting of SEQ ID NOs. 50 to 54 or a substantially similar sequence having at least 85%, 86%, 87%, 88%, 89%, 90%, 91%, 92%, 93%, 94%, 95%, 96%, 97%, 98%, or 99% sequence identity.

The present disclosure also provides a nucleic acid encoding the recombinant protein.

The present disclosure also provides a nucleic acid encoding the recombinant antibody or the fragment thereof.

The present disclosure also provides a pharmaceutical composition comprising an effective amount of the recombinant antibody or the fragment thereof and a pharmaceutically acceptable carrier for use in treating or detecting a disease. Alternatively, the present disclosure also provides a method for treating or detecting a disease in a subject in need of such treatment comprising administering a pharmaceutical composition comprising an effective amount of the recombinant antibody or the fragment thereof and a pharmaceutically acceptable carrier to the subject. Alternatively, the present disclosure also provides use of a pharmaceutical composition comprising an effective amount of the recombinant antibody or the fragment thereof and a pharmaceutically acceptable carrier in the manufacture of a medicament for treating or detecting disease.

Examples of the disease include, but are not limited to, a cancer, autoimmune and inflammatory disease, infectious disease, neurodegenerative disease or central nervous system disease.

Examples of the cancer include, but are not limited to, carcinoma, lymphoma, blastoma, sarcoma, and leukemia or lymphoid malignancies. More particular examples of such cancers include brain cancer, esophageal cancer, oral cavity cancer, squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In some embodiments of the disclosure, the cancer is a brain cancer.

Examples of the central nervous system disease include, but are not limited to, Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, or spinal muscular atrophy (SMA).

### BRIEF DESCRIPTION OF DRAWINGS

FIG. 1 shows the results of SDS-PAGE of VBT-engineered antibody variants.

FIGS. 2A to 2C show TfR1 binding affinities for the VBT-engineered antibody variants F1 (FIG. 2A), F2 (FIG. 2B) and F3 (FIG. 2C), respectively.

FIGS. 3A to 3C show the binding affinities of VBT-engineered antibody variants F1 (FIG. 3A), F2 (FIG. 3B) and F3 (FIG. 3C) to the Her2 protein.

FIG. 4A to 4B show antitumor activity of VBT-engineered antibody variants. FIG. 4A shows the result of percentage bioluminescence of antitumor activity in the orthotopic breast cancer model. FIG. 4B presents the percentage bioluminescence of antitumor activity results in the brain metastasis model.

FIGS. 5A and 5B show anti-tumor activity of VBT-engineered antibody variants compared with trastuzumab in an orthotopic and a brain metastasis model. FIG. 5A to FIG. 5B show the results for percentage of bioluminescence signal in mammary fat pad (FIG. 5A) and percentage of bioluminescence signal in intracranial (FIG. 5B).

FIG. 6 shows the ischemic constructions of F1 to F3.

### DETAILED DESCRIPTION OF THE INVENTION

Unless otherwise defined, scientific and technical terms used herein shall have the meanings that are commonly understood by those of ordinary skill in the art. Furthermore, unless otherwise required by context, singular terms shall include pluralities and plural terms shall include the singular. Generally, nomenclatures used in connection with, and techniques of, cell and tissue culture, molecular biology, and protein and oligo- or polynucleotide chemistry and hybridization described herein are those well-known and commonly used in the art.

The practice of the present disclosure may employ technologies comprising conventional techniques of cell biology, cell culture, antibody technology, and genetic engineering, which are within the ordinary skills of the art. Such techniques are explained fully in the literature.

As utilized in accordance with the present disclosure, the following terms, unless otherwise indicated, shall be understood to have the following meanings: The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually herein.

It must be noted that, as used in this specification and the appended claims, the singular forms “a,” “an,” and “the” include plural referents unless the content clearly dictates otherwise.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the term “recombinant protein” refers to a protein that is artificially engineered/constructed, rather than a naturally occurring protein. “Recombinant” contained in the term “recombinant protein” of the present disclosure does not denote a mode of production, and is merely used to indicate that “recombinant protein” does not naturally occur. The recombinant protein of the present disclosure may be an expressed protein and may be an assembled protein.

By “directly linked” is meant two sequences connected to each other without intervening or extraneous sequences. One of ordinary skill in the art will understand that “directly linked” encompasses the addition or removal of amino acids so long as the multimerization capacity is substantially unaffected.

The term “domain”, as used herein, refers to a linear molecular chain of amino acids that includes the amino acid sequence of an entire polypeptide or a portion of a polypeptide. Optionally, a domain may include one or more disulfide bonds or be chemically modified. Moreover, optionally a domain may comprise a non-proteinaceous element (such as a fluorophore). In one embodiment, however, the term “domain” does not comprise compounds that are chemically modified or comprise non-proteinaceous element(s).

A “functional domain”, as used herein, is a domain that is capable of fulfilling a certain function, such as specific binding to a certain binding partner or antigen, specific activation of a certain receptor, mediation of toxic effects, or fluorescence upon excitation with light of an appropriate wavelength.

The term “antibody” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin. In another aspect, the antibodies are polyclonal, monoclonal, bispecific, human, humanized or chimeric antibodies, single chain antibodies, Fv, Fab fragments, F(ab′) fragments, F(ab′)₂ fragments, fragments produced by a Fab expression library, anti-idiotypic (anti-Id) antibodies, CDR's, and epitope-binding fragments of any of the above which immunospecifically bind to cancer cell antigens, viral antigens or microbial antigens.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multi specific antibodies formed from antibody fragment(s).

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, (γ-carboxyglutamate), and O-phosphoserine. Amino acid analogs refer to compounds that have the same basic chemical structure as a naturally occurring amino acid, i.e., an a carbon that is linked to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As described herein, the terms “functionally equivalent” or “functionally equal” and “substantially similarity sequence” or “substantially similar” are used interchangeably herein. As applied to polypeptides, this means that when a protein sequence is optimally aligned with another (reference) protein sequence—such as through the programs GAP or BESTFIT using default gap weights—there is sequence identity in at least 81%, 82%, 83%, 84%, 85%, 86%, 87%, 88%, 89%, 90%, 91%, 92%, 93%, 94%, 95%, 96%, 97%, 98%, 99%, or 100% of amino acid residues to the entire sequence of said reference protein sequence. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups a are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutant thereof. Polypeptide sequences can also be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

The term “polynucleotide” or “nucleic acid,” as used interchangeably herein, refers to polymers of nucleotides of any length, and includes DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally occurring nucleotides with an analog, internucleotide modifications such as those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “a nucleic acid sequence encoding” a specified polypeptide refers to a nucleic acid sequence comprising the coding region of a gene or in other words the nucleic acid sequence which encodes a gene product. The coding region may be present in cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present disclosure may contain endogenous enhancers/promoters, splice junctions, intervening sequences, polyadenylation signals, etc. or a combination of both endogenous and exogenous control elements.

As used in the present disclosure, the term “pharmaceutical composition” means a mixture containing a therapeutic agent administered to a mammal, for example a human, for preventing, treating, or eliminating a particular disease or pathological condition that the mammal suffers.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include esophageal cancer, oral cavity cancer, squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In the context of cancer, the term “treating” includes any or all of: preventing growth of tumor cells, cancer cells, or of a tumor; preventing replication of tumor cells or cancer cells, lessening of overall tumor burden or decreasing the number of cancerous cells, and ameliorating one or more symptoms associated with the disease.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

The present disclosure provides a recombinant protein for introducing an additional domain in a protein. Accordingly, the present disclosure provides a recombinant protein comprising
a socket sequence comprising a pair of a socket upstream sequence and a socket downstream sequence; and
a plug sequence directly linked to the socket upstream sequence and socket downstream sequence in the pair.

In some embodiments of the disclosure, the plug sequence is flanked by the socket upstream sequence and the socket downstream sequence of the socket sequence providing an additional domain in the recombinant protein. Without wishing to be bound by theory, it is believed that the proline residues located at a terminal of the socket sequences are critical are critical for the successful introduction of the plug sequence as an additional domain. The side chain of proline is cyclic, which introduces a bending conformation into the polypeptide due to its restricted phi (@) angle. The inclusion of the proline residues is intended to provide a specific structural torsion angle for the inserted domain, i.e., the plug sequence, allowing it to orient outwardly, which is beneficial for its targeting function.

By applying these structural torsion angles, the configuration, tertiary structure, and function of the plug sequence are preserved without interference from the surrounding frame sequence. Conversely, the configuration, tertiary structure, and function of the frame sequence are also maintained without disruption from the inserted plug sequence. In some embodiments, depending on the characteristics of the plug sequence, if a torsion angle is required, a greater number of proline residues within the socket sequence may be employed; conversely, if a torsion angle is less critical, fewer proline residues are used. In some embodiments of the disclosure, the pair of the socket sequence comprises a total 1, 2, 3, 4, 5, 6, 7, or 8 proline residues.

In some embodiments of the disclosure, the socket sequence comprises a pair of a socket upstream sequence selected from the group consisting of amino acid sequences of SEQ ID NOs: 1 to 5 and a socket downstream sequence selected from the group consisting of amino acid sequences of SEQ ID NOs: 6 to 10. In some embodiments of the disclosure, the socket sequence comprises the pair of SEQ ID NOs: 1 and 6, 2 and 6, 3 and 6, 4 and 6, 5 and 6, 1 and 7, 2 and 7, 3 and 7, 4 and 7, 5 and 7, 1 and 8, 2 and 8, 3 and 8, 4 and 8, 5 and 8, 1 and 9, 2 and 9, 3 and 9, 4 and 9, 5 and 9, 1 and 10, 2 and 10, 3 and 10, 4 and 10, or 5 and 10. In some embodiments of the disclosure, the socket sequence comprises the pair of SEQ ID NOs: 11 and 16, 12 and 16, 13 and 16, 14 and 16, 15 and 16, 11 and 17, 12 and 17, 13 and 17, 14 and 17, 15 and 17, 11 and 18, 12 and 18, 13 and 18, 14 and 18, 15 and 18, 11 and 19, 12 and 19, 13 and 19, 14 and 19, 15 and 19, 11 and 20, 12 and 20, 13 and 20, 14 and 20, or 15 and 20.

In some embodiments of the disclosure, the plug sequence forms a functional domain. In some embodiments, the plug sequence relates to protein folding and structural organization. It is well established that most proteins fold into globular domains. Protein folding is predominantly driven by the hydrophobic effect, which functions to minimize the exposure of the polypeptide chain to the solvent environment. Typically, proteins adopt globular domain conformations characterized by minimal surface area. Peptides having molecular weights in the range of approximately 1 to 30 kDa generally fold into a single globular domain. In contrast, peptides exceeding approximately 50 kDa in molecular weight commonly form two or more independently folded domains. Notwithstanding the foregoing, certain proteins exhibit highly elongated conformations, which may comprise a linear arrangement of small globular domains or may be stabilized by specialized structural motifs.

In some embodiments of the disclosure, the plug sequence is a transporter peptide, which is based, at least in part, on the discovery that the transporter peptides can effectively bind to transferrin receptors (TfRs) on a cell. In the case of the cell being a cell of a tissue barrier, the binding of the transporter peptides to the TfRs induces transcytosis of the cell, thereby delivering the substance conjugated to the transporter peptides across the tissue barrier. In the case of the cell being the target cell, the binding of the transporter peptides to the TfRs directly deliver the substance to the target cell. Therefore, the transporter peptides of the present disclosure can serve as a drug delivery system. In particular, the transporter peptides of the present disclosure can be transported across a tissue barrier, especially a blood-brain barrier, by transcytosis and delivery the substance conjugated to the transporter peptides to brain. Therefore, the transporter peptides of the present invention can be used for prevention and/or treatment of CNS diseases.

In some embodiments, the plug sequence is a transporter peptide, consisting of 10 amino acids having the general sequence of

X₁-X₂-X₃-X₄-X₅-X₆-X₇-X₈-X₉-X₁₀

wherein,
X₁ is an acidic amino acid (Asp or Glu) or a polar uncharged amino acid (Asn, Gln, Cys, Ser, Thr, or Tyr);
X₂ is a nonpolar amino acid (Ile, Gly, Ala, Leu, Val, Pro, Phe, Trp, or Met);
X₃ is an arbitrary amino acid (Gly, Ala, Val, Leu, Ile, Phe, Tyr, Trp, His, Asp, Asn, Glu, Gln, Lys, Arg, Ser, Thr, Met, Cys, or Pro);
X₄ is an acidic amino acid (Asp or Glu) or a nonpolar amino acid (Ile, Gly, Ala, Leu, Val, Pro, Phe, Trp, or Met);
X₅ is a nonpolar amino acid (Ile, Gly, Ala, Leu, Val, Pro, Phe, Trp, or Met);
X₆ is an arbitrary amino acid (Gly, Ala, Val, Leu, Ile, Phe, Tyr, Trp, His, Asp, Asn, Glu, Gln, Lys, Arg, Ser, Thr, Met, Cys, or Pro);
X₇ is a basic amino acid (Lys, His, or Arg);
X₈ is an acidic amino acid (Asp or Glu) or a basic amino acid (Lys, His, or Arg);
X₉ is a nonpolar amino acid (Ile, Gly, Ala, Leu, Val, Pro, Phe, Trp, or Met) or a polar uncharged amino acid (Asn, Gln, Cys, Ser, Thr, or Tyr); and
X₁₀ is an acidic amino acid (Asp or Glu) or a polar uncharged amino acid (Asn, Gln, Cys, Ser, Thr, or Tyr).

In some embodiments,
X₁ is Asp, Glu, Asn, Gln, Cys, Ser, Thr, or Tyr;
X₂ is Ile, Gly, Ala, Leu, Val, Pro, Phe, Trp, or Met;
X₃ is Gly, Ala, Val, Leu, Ile, Phe, Tyr, Trp, His, Asp, Asn, Glu, Gln, Lys, Arg, Ser, Thr, Met, Cys, or Pro;
X₄ is Val, Asp, Glu, Ile, Gly, Ala, Leu, Pro, Phe, Trp, or Met;
X₅ is Leu, Ile, Gly, Ala, Val, Pro, Phe, Trp, or Met;
X₆ is Gly, Ala, Val, Leu, Ile, Phe, Tyr, Trp, His, Asp, Asn, Glu, Gln, Lys, Arg, Ser, Thr, Met, Cys, or Pro;
X₇ is Lys, His or Arg;
X₈ is Asp, Glu, Lys, His, or Arg;
X₉ is Ile, Gly, Ala, Leu, Val, Pro, Phe, Trp, Met, Asn, Gln, Cys, Ser, Thr, or Tyr; and
X₁₀ is Asp, Glu, Asn, Gln, Cys, Ser, Thr, or Tyr.

In some embodiments of the disclosure, the plug sequence is selected from the group consisting of SEQ ID NOs. 21 to 49.

In some embodiments of the disclosure, the socket sequence and plug sequence are integrated into the frame sequence or linked to a terminal of the frame sequence.

In some embodiments of the disclosure, the recombinant protein further comprises a frame sequence. Examples of the frame sequence include, but are not limited to, a structural protein, enzymatic protein, transport protein, storage protein, signaling protein, defensive protein, contractile protein, and regulatory protein. In some embodiments, the frame sequence is an enzyme, receptor, ligand, gate, messenger, cytokine, antibiotics, hormone (such as insulin), antigen, or antibody. In some further embodiments, the frame sequence is an antibody or a fragment thereof. Examples of the antibody include, but are not limited to trastuzumab, rituximab, bevacizumab, cetuximab, nivolumab, pembrolizumab, ipilimumab, adalimumab, infliximab, tocilizumab, natalizumab, ustekinumab, palivizumab, sotrovimab, omalizumab, or denosumab. In some embodiments, the antibody is trastuzumab.

In some embodiments of the disclosure, the recombinant protein is conjugated with a therapeutic agent. In some embodiments of the disclosure, the recombinant protein, which is chemically or biologically linked to a radioactive agent, a cytokine, an interferon, a target or reporter moiety, an enzyme, a peptide or protein or a therapeutic agent. Examples of the therapeutic agent include, but are not limited to antimetabolites, alkylating agents, alkylating-like agents, DNA minor groove alkylating agents, anthracyclines, antibiotics, calicheamicins, antimitotic agents, topoisomerase inhibitors, proteasome inhibitors, cytokine, interferon, and radioisotopes.

In some embodiments of the disclosure, the frame sequence is an antibody or a fragment thereof, and the socket sequence and plug sequence are integrated into a hinge region, linked to a terminal of a heavy chain or linked to a terminal of a light chain of the antibody or the fragment thereof.

In some embodiments of the disclosure, the recombinant protein comprises the sequence selected from the group consisting of SEQ ID NOs. 50 to 54 or a substantially similar sequence having at least 85% sequence identity.

The sequence listing is shown in Table 1.

| | SEQ ID | |
|---|---|---|
| | NO | Sequence |
| Socket upstream | 1 | CGTGAPP |
| sequence 1-1 | | |
| Socket upstream | 2 | CGTGAP |
| sequence 1-2 | | |
| Socket upstream | 3 | CGTGAPPP |
| sequence 1-3 | | |
| Socket upstream | 4 | CGTGAPPPP |
| sequence 1-4 | | |
| Socket upstream | 5 | CGTGA |
| sequence 1-5 | | |
| Socket downstream | 6 | PSGAC |
| sequence 1-1 | | |
| Socket downstream | 7 | PPSGAC |
| sequence 1-2 | | |
| Socket downstream | 8 | PPPSGAC |
| sequence 1-3 | | |
| Socket downstream | 9 | PPPPSGAC |
| sequence 1-4 | | |
| Socket downstream | 10 | SGAC |
| sequence 1-5 | | |
| Socket upstream | 11 | SGAGGTGAPP |
| sequence 2-1 | | |
| Socket upstream | 12 | SGAGGTGAP |
| sequence 2-2 | | |
| Socket upstream | 13 | SGAGGTGAPPP |
| sequence 2-3 | | |
| Socket upstream | 14 | SGAGGTGAPPPP |
| sequence 2-4 | | |
| Socket upstream | 15 | SGAGGTGA |
| sequence 2-5 | | |
| Socket downstream | 16 | PSGASGAG |
| sequence 2-1 | | |
| Socket downstream | 17 | PPSGASGAG |
| sequence 2-2 | | |
| Socket downstream | 18 | PPPSGASGAG |
| sequence 2-3 | | |
| Socket downstream | 19 | PPPPSGASGAG |
| sequence 2-4 | | |
| Socket downstream | 20 | SGASGAG |
| sequence 2-5 | | |
| PT-034 | 21 | DISVLMKEYQ |
| PT-001 | 22 | DISVLMKEYD |
| PT-004 | 23 | DIDVLMKEYD |
| PT-008 | 24 | DIHVLMKEYD |
| PT-011 | 25 | DILVLMKEYD |
| PT-014 | 26 | DIPVLMKEYD |
| PT-017 | 27 | DITVLMKEYD |
| PT-022 | 28 | DISLLMKEYD |
| PT-023 | 29 | DISVIMKEYD |
| PT-024 | 30 | DISVVMKEYD |
| PT-025 | 31 | DISVLDKEYD |
| PT-026 | 32 | DISVLNKEYD |
| PT-027 | 33 | DISVLCKEYD |
| PT-028 | 34 | DISVLKKEYD |
| PT-029 | 35 | DISVLEKEYD |
| PT-030 | 36 | DISVLQKEYD |
| PT-031 | 37 | DISVLMKEYN |
| PT-033 | 38 | DISVLMKEYE |
| PT-051 | 39 | DISVLSKEYD |
| PT-054 | 40 | DISVLMKDYD |
| PT-055 | 41 | DISVLMKEFD |
| PT-056 | 42 | DISVLMKESD |
| PT-057 | 43 | DISVLMKETD |
| PT-062 | 44 | NISVLMKEYD |
| PT-063 | 45 | DISELMKEYD |
| PT-064 | 46 | DISVLLKEYD |
| PT-065 | 47 | DISVLMKHYD |
| PT-066 | 48 | DITVLCKEYN |
| PT-067 | 49 | DIHVLDKEYD |
| Insert-1 | 50 | CGTGAPPDISVLMKEYQPSGAC |
| Insert-2 | 51 | SGAGGTGAPPDISVLMKEYQPSGASGAG |
| Trastuzumab-insert 1 | 52 | EVQLVESGGG LVQPGGSLRL SCAASGFNIK DTYIHWVRQA PGKGLEWVAR |
| | | IYPTNGYTRY ADSVKGRFTI SADTSKNTAY LQMNSLRAED TAVYYCSRWG |
| | | GDGFYAMDYW GQGTLVTVSS ASTKGPSVFP LAPSSKSTSG GTAALGCLVK |
| | | DYFPEPVTVS WNSGALTSGV HTFPAVLQSS GLYSLSSVVT VPSSSLGTQT |
| | | YICNVNHKPS NTKVDKKVEP KSCDK CGTGAPPDISVLMKEYQPSGAC THTCP |
| | | PCPAPELLGG PSVFLFPPKP KDTLMISRTP EVTCVVVDVS HEDPEVKFNW |
| | | YVDGVEVHNA KTKPREEQYN STYRVVSVLT VLHQDWLNGK EYKCKVSNKA |
| | | LPAPIEKTIS KAKGQPREPQ VYTLPPSREE MTKNQVSLTC LVKGFYPSDI |
| | | AVEWESNGQP ENNYKTTPPV LDSDGSFFLY SKLTVDKSRW QQGNVFSCSV |
| | | MHEALHNHYT QKSLSLSPG |
| Trastuzumab-insert 2 | 53 | EVQLVESGGG LVQPGGSLRL SCAASGFNIK DTYIHWVRQA PGKGLEWVAR |
| | | IYPTNGYTRY ADSVKGRFTI SADTSKNTAY LQMNSLRAED TAVYYCSRWG |
| | | GDGFYAMDYW GQGTLVTVSS ASTKGPSVFP LAPSSKSTSG GTAALGCLVK |
| | | DYFPEPVTVS WNSGALTSGV HTFPAVLQSS GLYSLSSVVT VPSSSLGTQT |
| | | YICNVNHKPS NTKVDKKVEP KSCDK SGAGGTGAPP DISVLMKEYQ |
| | | PSGASGAG THTCP PCPAPELLGG PSVFLFPPKP KDTLMISRTP |
| | | EVTCVVVDVS HEDPEVKFNW YVDGVEVHNA KTKPREEQYN STYRVVSVLT |
| | | VLHQDWLNGK EYKCKVSNKA LPAPIEKTIS KAKGQPREPQ VYTLPPSREE |
| | | MTKNQVSLTC LVKGFYPSDI AVEWESNGQP ENNYKTTPPV LDSDGSFFLY |
| | | SKLTVDKSRW QQGNVFSCSV MHEALHNHYT QKSLSLSPG |
| Trastuzumab-insert 3 | 54 | DIQMTQSPSSLSASVGDRVTITCRASQDVNTAVAWYQQKPGKAPKLLIYSASFL |
| | | YSGVPSRFSGSRSGTDFTLTISSLQPEDFATYYCQQHYTTPPTFGQGTKVEIKRT |
| | | VAAPSVFIFPPSDEQLKSGTASVVCLLNNFYPREAKVQWKVDNALQSGNSQES |
| | | VTEQDSKDSTYSLSSTLTLSKADYEKHKVYACEVTHQGLSSPVTKSFNRGEC |
| | | SGAGGTGAPPDISVLMKEYQPSGASGAG |

The present disclosure also provides a recombinant antibody or a fragment thereof, comprising
a socket sequence comprising
   a pair of a socket upstream sequence selected from the group consisting of amino acid sequences of SEQ ID NOs: 1 to 5 and a socket downstream sequence selected from the group consisting of amino acid sequences of SEQ ID NOs: 6 to 10; or
   a pair of a socket upstream sequence selected from the group consisting of amino acid sequences of SEQ ID NOs: 11 to 15 and a socket downstream sequence selected from the group consisting of amino acid sequences of SEQ ID NOs: 16 to 20;
a plug sequence selected from the group consisting of SEQ ID NOs. 21 to 49 and directly linked to the socket upstream sequence and socket downstream sequence in the pair; and
a frame sequence of an antibody or a fragment thereof;
wherein the socket sequence and plug sequence are integrated into a hinge region, linked to a terminal of a heavy chain or linked to a terminal of a light chain of the antibody or the fragment thereof.

The present disclosure also provides a nucleic acid encoding the recombinant protein or recombinant antibody or fragment thereof.

The recombinant protein as disclosed herein may be produced by any suitable method, many of which are known to those skilled in the art. For example, the recombinant protein may be chemically synthesized, or produced using recombinant DNA technology (e.g., in bacterial cells, in cell culture (mammalian, yeast or insect cells), in plants or plant cells, or by cell-free prokaryotic or eukaryotic-based expression systems, by other in vitro systems, etc.). In some embodiments, the present disclosure provides a vector comprising the nucleic acid molecule of the present disclosure. In one embodiment, the recombinant protein may be expressed using a vector, wherein the nucleic acid molecule encoding said recombinant protein is operably linked to a promoter sequence. In one embodiment, the promoter is a constitutive promoter. In another embodiment, the promoter is an inducible promoter.

The present disclosure also provides a pharmaceutical composition comprising the recombinant protein and optionally a pharmaceutically acceptable carrier or excipient.

The pharmaceutical compositions of the disclosure are formulated with suitable carriers, excipients, and other agents that provide improved transfer, delivery, tolerance, and the like. A multitude of appropriate formulations can be found in the formulary known to all pharmaceutical chemists. These formulations include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions carbowax (polyethylene glycols of various molecular weights), semi-solid gels, and semi-solid mixtures containing carbowax.

The dose of recombinant protein administered to a patient may vary depending upon the age and the weight of the patient, target disease, conditions, route of administration, and the like. The preferred dose is typically calculated according to body weight or body surface area. When a recombinant protein of the present disclosure is used for treating a tumor in an adult patient, it may be advantageous to intravenously administer the recombinant protein of the present disclosure. Depending on the severity of the condition, the frequency and the duration of the treatment can be adjusted. Effective dosages and schedules for administering the recombinant protein may be determined empirically; for example, patient progress can be monitored by periodic assessment, and the dose adjusted accordingly. Moreover, interspecies scaling of dosages can be performed using well-known methods in the art.

Various delivery systems are known and can be used to administer the pharmaceutical composition of the disclosure, e.g., encapsulation in liposomes, microparticles, microcapsules, recombinant cells capable of expressing the mutant viruses, or receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, intra-arterial, intra-thecal, intra-vesical, or intratumoral, epidural, oral routes or external routes. The composition may be administered by any convenient route, for example by infusion or bolus injection, by absorption through epithelial or mucocutaneous linings (e.g., oral mucosa, rectal and intestinal mucosa, etc.), and may be administered together with other biologically active agents. Administration can be systemic or local.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Disclosures of Controlled Release, Langer and Wise (eds.), 1974, CRC Pres., Boca Raton, Fla. In yet another embodiment, a controlled release system can be placed in proximity to the composition's target, thus requiring only a fraction of the systemic dose (see, e.g., Goodson, 1984, in Medical Disclosures of Controlled Release, supra, vol. 2, pp. 115-138). Other controlled release systems are discussed in the review by Langer, 1990, Science 249:1527-1533.

In some embodiments, the pharmaceutical composition can be formulated in various ways, according to the corresponding route of administration, such as a liquid solution, a suspension, an emulsion, a syrup, a tablet, a pill, a capsule, a sustained release formulation, a powder, a granule, an ampoule, an injection, an infusion, a kit, an ointment, a lotion, a liniment, a cream or a combination thereof. If necessary, it may be sterilized or mixed with any pharmaceutically acceptable carrier or excipient, many of which are known to one of ordinary skill in the art.

The external route as used herein is also known as local administration, which includes but is not limited to administration by insufflation and inhalation. Examples of various types of preparation for local administration include ointments, lotions, creams, gels, foams, preparations for delivery by transdermal patches, powders, sprays, aerosols, capsules or cartridges for use in an inhaler or insufflator or drops (e.g., eye or nose drops), solutions/suspensions for nebulisation, suppositories, pessaries, retention enemas and chewable or suckable tablets or pellets or liposome or microencapsulation preparations.

The present disclosure also provides a pharmaceutical composition comprising an effective amount of the recombinant antibody or the fragment thereof and a pharmaceutically acceptable carrier for use in treating or detecting a disease. Alternatively, the present disclosure also provides a method for treating or detecting a disease in a subject in need of such treatment comprising administering a pharmaceutical composition comprising an effective amount of the recombinant antibody or the fragment thereof and a pharmaceutically acceptable carrier to the subject. Alternatively, the present disclosure also provides use of a pharmaceutical composition comprising an effective amount of the recombinant antibody or the fragment thereof and a pharmaceutically acceptable carrier in the manufacture of a medicament for treating or detecting disease.

The recombinant protein of the present disclosure may also be used to detect and/or measure a cancer, e.g., for diagnostic purposes. Exemplary diagnostic assays for the recombinant protein may comprise, e.g., contacting a sample, obtained from a patient, with the recombinant protein of the disclosure, wherein the recombinant protein is labeled with a detectable label or reporter molecule. Alternatively, an unlabeled recombinant protein can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure coronavirus in a sample include enzyme-linked immunosorbent assay (ELISA), radioimmunoassay (RIA), and fluorescence-activated cell sorting (FACS).

Examples of the disease include, but are not limited to, a cancer, autoimmune and inflammatory disease, infectious disease or neurodegenerative disease and central nervous system disease. In some further embodiments, examples of the disease include, but are not limited to, cancer, rheumatoid arthritis, psoriasis, Crohn's disease, ulcerative colitis, giant cell arteritis, multiple sclerosis, respiratory syncytial virus infection, or SARS-COV-2 infection, allergic asthma, chronic idiopathic urticaria, osteoporosis, or bone metastases, Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

Examples of cancer include, but are not limited to, carcinoma, lymphoma, blastoma, sarcoma, and leukemia or lymphoid malignancies. More particular examples of such cancers include brain cancer, esophageal cancer, oral cavity cancer, squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In some embodiments of the disclosure, the cancer is a brain cancer.

The following examples are provided to aid those skilled in the art in practicing the present disclosure.

### EXAMPLES

### Example 1 VBT-DNA-Engineering Trastuzumab

### Materials and Methods

### Construction and Protein Preparation

To create a VBT-trastuzumab IgG1 expression construct, the antigen-binding sites of trastuzumab VH and VL were cloned into a pIgG expression vector. Subsequently, a modified VBT DNA sequence was inserted to produce VBT-engineered antibody variant expression plasmids. All procedures were performed using primer-overlap PCR extension and an infusion enzyme protocol.

VBT-trastuzumab IgG protein was produced in Expi293F cells via transient transfection using plasmids encoding the VH and VL regions. Thirty milliliters of transfected Expi293F cells (INVITROGEN™) were harvested on Day 5. The secreted IgG protein in the culture medium was loaded onto GENSCRIPT™ Protein A Resin FF (Cat. No. L00464-100) and washed with PBS. The IgG protein was eluted using 0.1 M glycine-HCl and subsequently buffer-exchanged into PBS (INVITROGEN™, pH 7.4). The protein solution was filtered through a 0.2 μm syringe filter and stored at 4° C. until further use.

### SDS-PAGE Analysis

The VBT-DNA-engineering trastuzumab was analyzed for integrity by SDS-PAGE. It was performed using INVITROGEN™ NuPAGE® electrophoresis system according to the manufacturer's protocols. Protein analyzed on the SDS-PAGE gels using 12% NuPAGE® Bis-Tris gel. For sample loading, 5 μg of each preparation was applied per lane for reduction while 2.5 μg of each preparation was applied per lane for non-reduction. Gels were stained with Coomassie Blue for analysis.

### Results

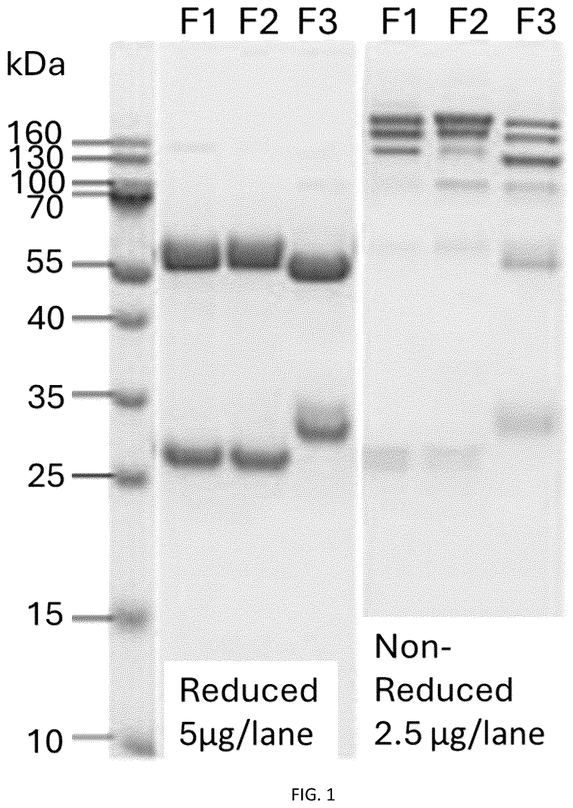

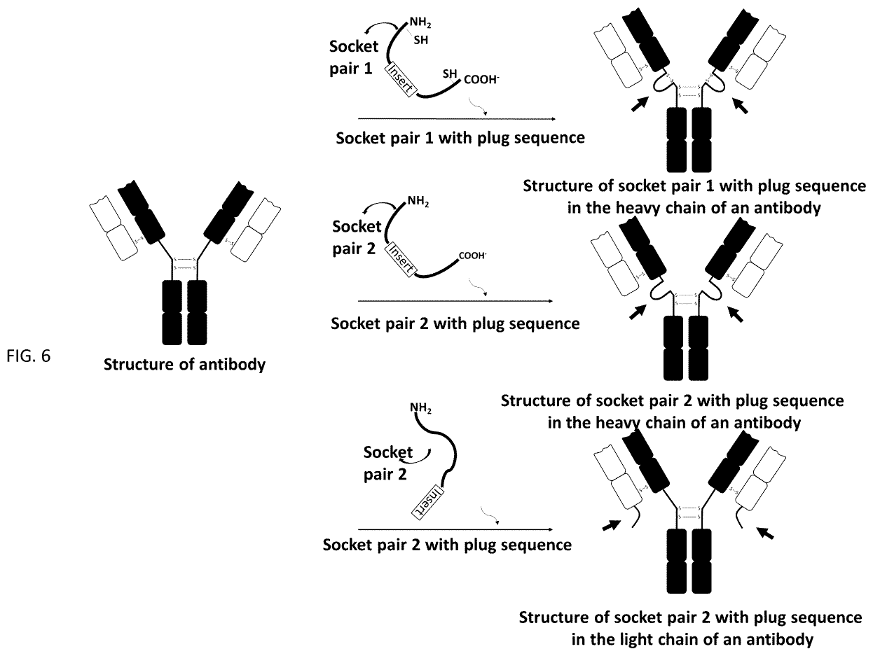

All expressed proteins were confirmed by protein sequencing. SDS-PAGE analysis showed that all VBT-engineered antibody variants migrated at their expected molecular weights, approximately 150 kDa (FIG. 1). The heavy chain corresponds to approximately 50 kDa, while the light chain corresponds to approximately 25 kDa. F1 refers to the trastuzumab with its heavy chain engineered with SEQ ID NO. 52. F2 refers to the trastuzumab with its heavy chain engineered with SEQ ID NO. 53. F3 refers to the trastuzumab with its light chain engineered with SEQ ID NO. 54. The ischemic constructions of F1 to F3 are shown in FIG. 6.

### Example 2 Binding Ability Assay of the VBT-DNA Engineering Trastuzumab to TfR Protein

### Materials and Methods

To determine the affinity of the targeting peptide to TfR1, a fiber optic particle plasmon resonance (FOPPR) affinity study was performed according to the manufacturer's instruction. Briefly, 80 μL of TfR1 (10 μg/ml) (ACRO BIOSYSTEMS™. Cat. No. CD1-H5243) dissolved in PBS buffer (pH 7.4) was pipetted into the EDC/NHS preactivated surface of standard chip NANOAU-MM™ for protein conjugating to the carboxyl group of the surface sensor chip. F1, F2 and F3 proteins were loading to each chip with the concentrations of 1.56 ng/ml, 3.125 ng/ml, 6.25 ng/ml, 12.5 ng/ml, 25 ng/ml, and 50 ng/ml, respectively. The equilibrium dissociation constant (KD) of the molecules was determined from the association and dissociation rate constants by analyzing changes in light intensity.

### Results

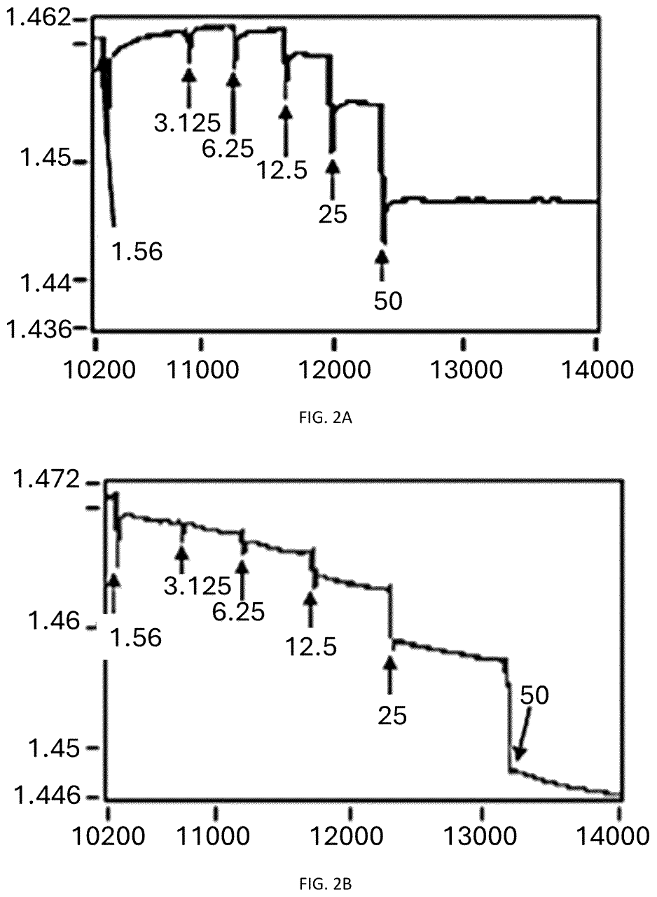

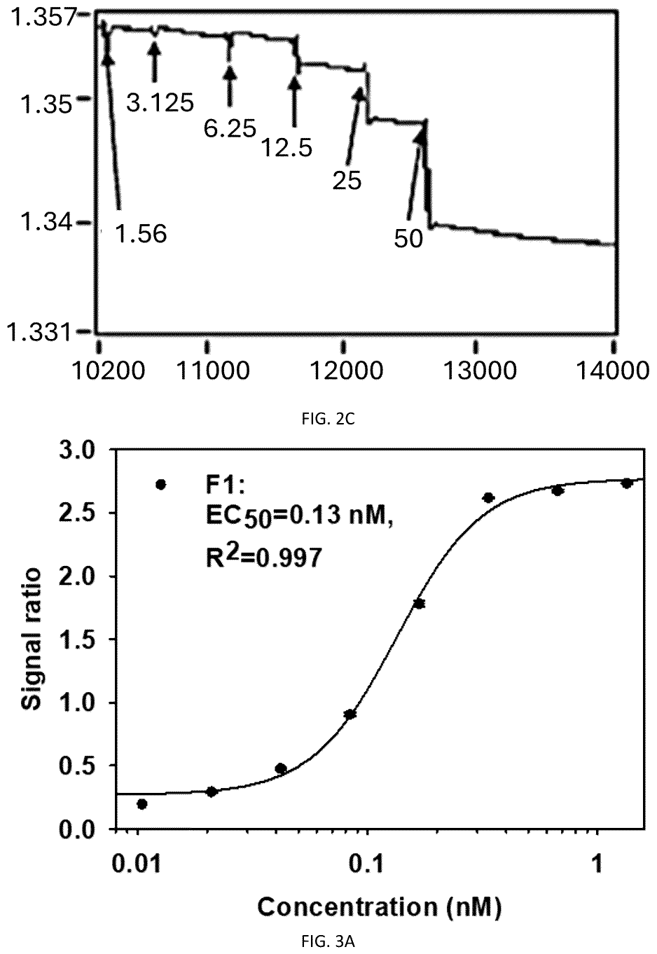

The binding affinity results indicated that F1, F2, and F3 have dissociation constants (KD) for TfR1 of 275.57 nM, 258.31 nM, and 123.24 nM, respectively (FIGS. 2A to 2C). The binding affinity assay for trastuzumab to HER2 was conducted by FOPPR assay as F1, F2 and F3. There is no observation of interaction between trastuzumab and TfR1 molecule within the concentrations from 0.56 ng/mL to 50 ng/ml, which could not generate the value of KD.

### Example 3 Binding Ability Assay of the VBT-DNA Engineering Trastuzumab to Her2 Protein

### Materials and Methods

To determine Her2 protein binding, the Trastuzumab Pharmacokinetic ELISA Kit (Cat. No. EL-1611-201, AFFINITYIMMUNO™ Inc) were used according to the manufacturer's protocols. Perform 2 folds serial dilutions of test samples from 200 ng/ml to 1.56 ng/mL. Added 100 μl of diluted test samples to wells on the plate. Incubate for 1 hour at room temperature on a plate shaker at 300 rpm. Discard the content of the plate and wash the wells three times with 200 μl 1× wash buffer per well. Added 100 μL detection reagent to wells on the plate. Incubate for 1 hour at room temperature on a plate shaker at 300 rpm. Discard the content of the plate and wash the wells three times with 200 μL 1× wash buffer per well. Added 100 μL of TMB to each well on the plate. Incubate for 6-10 minutes at room temperature protected from light. Added 100 μL of TMB stop solution to each well on plate. Determine absorbance with a microplate reader at 450 nm against 620 nm. The EC50 were calculated by fitting a 4-parameter logistic curve in SigmaPlot10.0.

### Results

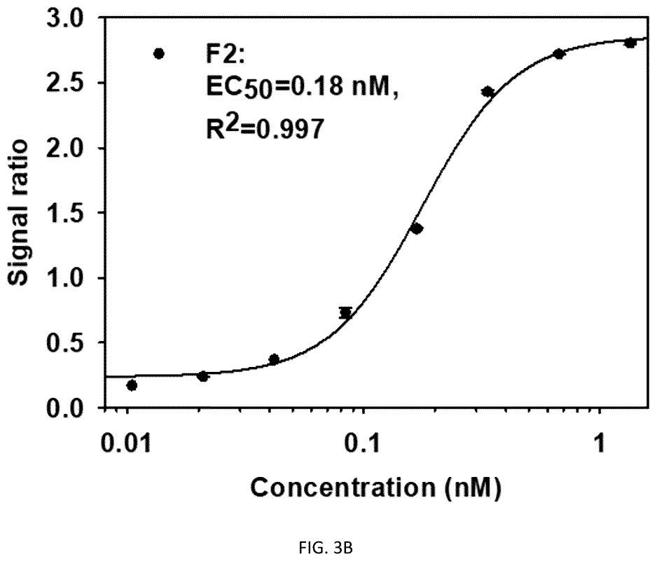

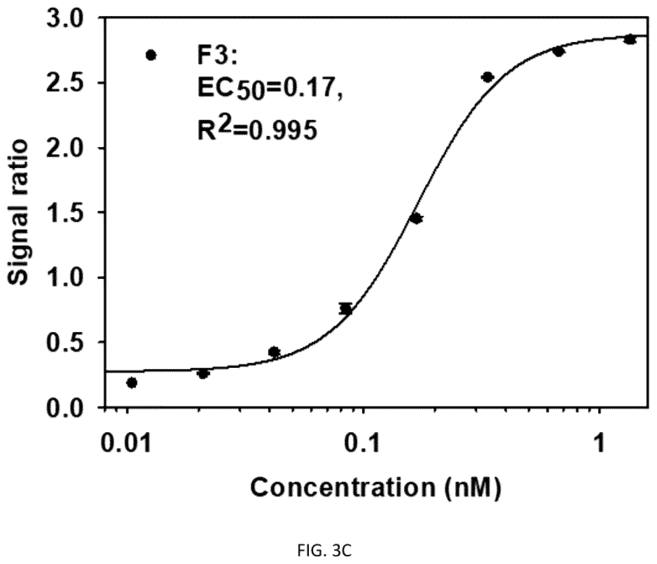

The binding affinity results indicated that F1, F2, and F3 have half maximal effective concentration (EC50) for Her2 protein of 0.13 nM, 0.18 nM, and 0.17 nM, respectively (FIGS. 3A to 3C). The EC50 of trastuzumab binding to Her2 receptor determined by ELISA ranges from 0.11 nM to 0.18 nM (Table 2).

TABLE 2
Binding affinities of Trastuzumab antibody to Her2 protein.

| | Assay 1 | | Assay 2 | | Assay 3 | |
|---|---|---|---|---|---|---|
| | nM | O.D.* | nM | O.D.* | nM | O.D.* |
| | 0.717 | 1.193 | 0.717 | 1.648 | 0.690 | 2.907 |
| | 0.179 | 0.878 | 0.359 | 1.42 | 0.345 | 2.556 |
| | 0.045 | 0.281 | 0.179 | 1.075 | 0.173 | 1.504 |
| | 0.011 | 0.099 | 0.090 | 0.671 | 0.086 | 0.764 |
| | | | 0.045 | 0.374 | 0.043 | 0.392 |
| | | | 0.022 | 0.204 | 0.022 | 0.183 |
| EC_{50 (nM)} | | | 0.011 | 0.132 | 0.011 | 0.103 |

| | | |
|---|---|---|
| 0.112 | 0.147 | 0.182 |

*O.D.: Optical density; the ELISA readout is measured as the absorbance at 450 nm minus the absorbance at 620 nm.

### Example 4 Anti-Tumor Activity of VBT-DNA Engineered Trastuzumab

### Materials and Methods

The anti-tumor activity of VBT-DNA engineered trastuzumab was studied in an animal model comprising orthotopic breast cancer and brain metastases. Twenty NOD SCID mouse (BIOLASCO™) at age of 6-8 weeks were divided into vehicle (PBS) group (n=4), F1 group (n=4), F2 group (n=4), and F3 (n=4).

All animals were implanted subcutaneously with E2 pellets 28 days prior. BT474-Luc cells (5×10⁶ cells in 100 μl per mouse) were inoculated into the mammary fat pad 21 days prior, and BT474-Luc cells (5×10⁴ cells in 4 μL per mouse) were inoculated intracranially.

Beside vehicle (PBS) control, each group was administrated with 4 mg/kg of the tested protein intraperitoneal injection biweekly for 4 weeks. Termination of animal study was conducted by Day 34.

In this example, tumor burden was measured, and mortality was monitored.

### Results

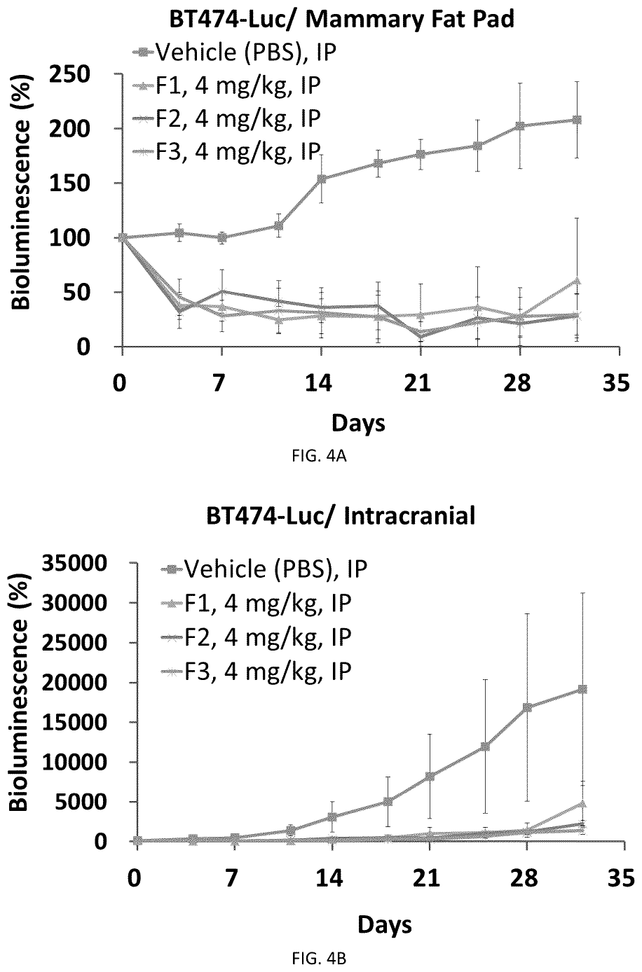

The data showed that the anti-tumor activities of F1, F2, and F3 were effective on orthotopic breast cancer and brain metastasis models, compared to the vehicle group, which received no drug treatment (FIGS. 4A to 4B).

### Example 5 Anti-Tumor Activity of VBT-DNA Engineered Trastuzumab Compared to Unmodified Trastuzumab in Low Dose

### Materials and Methods

The anti-tumor activity of VBT-DNA engineered trastuzumab was studied in an animal model comprising orthotopic breast cancer and brain metastases. Three NOD SCID mouse (BioLASCO™) at age of 6-8 weeks were divided into trastuzumab (n=1), F1 group (n=1), and F2 group (n=1).

All animals were implanted subcutaneously with E2 pellets 28 days prior. BT474-Luc cells (5×10⁶ cells in 100 μL per mouse) were inoculated into the mammary fat pad 21 days prior, and BT474-Luc cells (5×10⁴ cells in 4 μL per mouse) were inoculated intracranially.

Each group was administrated with 1 mg/kg of the tested protein intraperitoneal injection on Day 35. Termination of animal study was conducted by Day 46.

In this example, tumor burden was measured, and mortality was monitored.

### Results

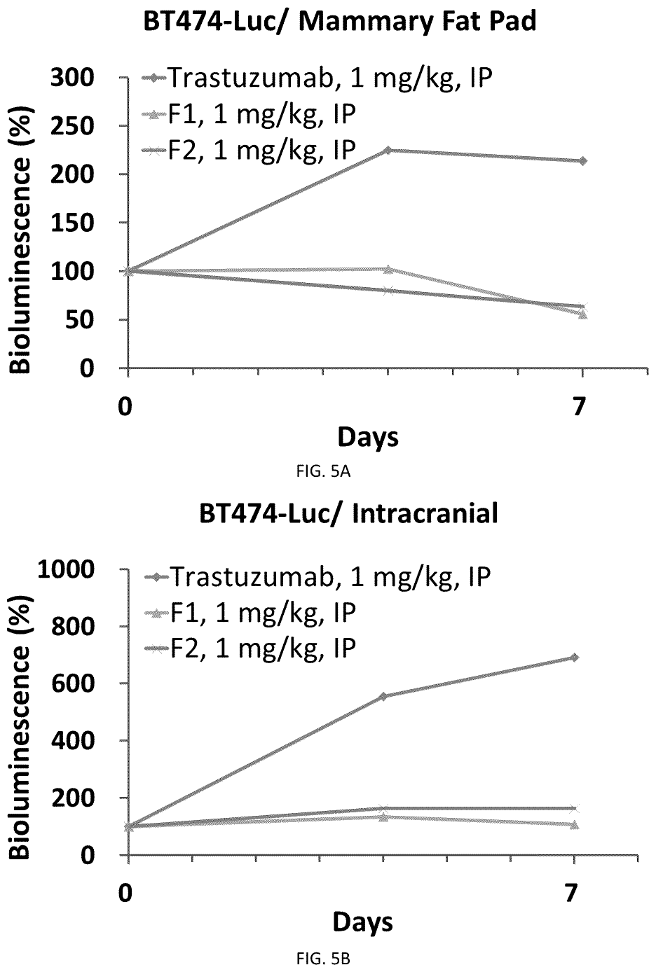

The data showed that the anti-tumor activities of F1, F2 were effective on orthotopic breast cancer and brain metastasis models, compared to the trastuzumab group (FIGS. 5A and 5B).

While the present invention has been described in conjunction with the specific embodiments set forth above, many alternatives thereto and modifications and variations thereof will be apparent to those of ordinary skill in the art. All such alternatives, modifications and variations are regarded as falling within the scope of the present invention.

1. A recombinant protein comprising
a socket sequence comprising
a pair of a socket upstream sequence selected from the group consisting of amino acid sequences of SEQ ID NOs: 1 to 5 and a socket downstream sequence selected from the group consisting of amino acid sequences of SEQ ID NOs: 6 to 10; or
a pair of a socket upstream sequence selected from the group consisting of amino acid sequences of SEQ ID NOs: 11 to 15 and a socket downstream sequence selected from the group consisting of amino acid sequences of SEQ ID NOs: 16 to 20; and
a plug sequence directly linked to the socket upstream sequence and socket downstream sequence in the pair.

2. The recombinant protein of claim 1, wherein the plug sequence forms a functional domain.

3. The recombinant protein of claim 1, wherein the plug sequence comprises a size of no more than 50 kDa.

4. The recombinant protein of claim 1, wherein the plug sequence comprises a size of about 1 kDa to about 30 kDa.

5. The recombinant protein of claim 1, wherein the plug sequence is selected from the group consisting of SEQ ID NOs. 21 to 49.

6. The recombinant protein of claim 1, which comprises the sequence selected from the group consisting of SEQ ID NOs. 50 to 54 or a substantially similar sequence having at least 85% sequence identity.

7. The recombinant protein of claim 1, which further comprises a frame sequence.

8. The recombinant protein of claim 7, wherein the socket sequence and plug sequence are integrated into the frame sequence or linked to a terminal of the frame sequence.

9. The recombinant protein of claim 7, wherein the frame sequence is an antibody or a fragment thereof.

10. The recombinant protein of claim 9, wherein the socket sequence and plug sequence are integrated into a hinge region, linked to a terminal of a heavy chain or linked to a terminal of a light chain of the antibody or the fragment thereof.

11. The recombinant protein of claim 9, wherein the antibody is trastuzumab.

12. A recombinant antibody or a fragment thereof, comprising
a socket sequence comprising
a pair of a socket upstream sequence selected from the group consisting of amino acid sequences of SEQ ID NOs: 1 to 5 and a socket downstream sequence selected from the group consisting of amino acid sequences of SEQ ID NOs: 6 to 10; or
a pair of a socket upstream sequence selected from the group consisting of amino acid sequences of SEQ ID NOs: 11 to 15 and a socket downstream sequence selected from the group consisting of amino acid sequences of SEQ ID NOs: 16 to 20;
a plug sequence selected from the group consisting of SEQ ID NOs. 21 to 49 and directly linked to the socket upstream sequence and socket downstream sequence in the pair; and
a frame sequence of an antibody or a fragment thereof;
wherein the socket sequence and plug sequence are integrated into a hinge region, linked to a terminal of a heavy chain or linked to a terminal of a light chain of the antibody or the fragment thereof.

13. The recombinant antibody or the fragment thereof of claim 12, wherein the antibody is trastuzumab.

14. The recombinant antibody or the fragment thereof of claim 12, which comprises the sequence selected from the group consisting of SEQ ID NOs. 50 to 54 or a substantially similar sequence having at least 85% sequence identity.

15. A nucleic acid encoding the recombinant protein of claim 1.

16. A nucleic acid encoding the recombinant antibody or the fragment thereof of claim 12.

17. A method for treating or detecting a disease in a subject in need of such treatment, comprising administering a pharmaceutical composition comprising an effective amount of the recombinant protein of claim 1 and a pharmaceutically acceptable carrier to the subject.

18. The method of claim 17, wherein the disease is a cancer, autoimmune and inflammatory disease, infectious disease, neurodegenerative disease or central nervous system disease.

19. The method of claim 18, wherein the cancer is brain cancer, esophageal cancer, oral cavity cancer, squamous cell cancer, lung cancer, cancer of the peritoneum, hepatocellular cancer, gastric or stomach cancer, pancreatic cancer, glioma, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, or head and neck cancer.

20. A method for treating or detecting a disease in a subject in need of such treatment comprising administering a pharmaceutical composition comprising an effective amount of the recombinant antibody or the fragment thereof of claim 12 and a pharmaceutically acceptable carrier to the subject.


SEQUENCE LISTING (54 sequences)

SEQ ID NO: 1 - protein, 7 aa, synthetic construct
CGTGAPP

SEQ ID NO: 2 - protein, 6 aa, synthetic construct
CGTGAP

SEQ ID NO: 3 - protein, 8 aa, synthetic construct
CGTGAPPP

SEQ ID NO: 4 - protein, 9 aa, synthetic construct
CGTGAPPPP

SEQ ID NO: 5 - protein, 5 aa, synthetic construct
CGTGA

SEQ ID NO: 6 - protein, 5 aa, synthetic construct
PSGAC

SEQ ID NO: 7 - protein, 6 aa, synthetic construct
PPSGAC

SEQ ID NO: 8 - protein, 7 aa, synthetic construct
PPPSGAC

SEQ ID NO: 9 - protein, 8 aa, synthetic construct
PPPPSGAC

SEQ ID NO: 10 - protein, 4 aa, synthetic construct
SGAC

SEQ ID NO: 11 - protein, 10 aa, synthetic construct
SGAGGTGAPP

SEQ ID NO: 12 - protein, 9 aa, synthetic construct
SGAGGTGAP

SEQ ID NO: 13 - protein, 11 aa, synthetic construct
SGAGGTGAPPP

SEQ ID NO: 14 - protein, 12 aa, synthetic construct
SGAGGTGAPPPP

SEQ ID NO: 15 - protein, 8 aa, synthetic construct
SGAGGTGA

SEQ ID NO: 16 - protein, 8 aa, synthetic construct
PSGASGAG

SEQ ID NO: 17 - protein, 9 aa, synthetic construct
PPSGASGAG

SEQ ID NO: 18 - protein, 10 aa, synthetic construct
PPPSGASGAG

SEQ ID NO: 19 - protein, 11 aa, synthetic construct
PPPPSGASGAG

SEQ ID NO: 20 - protein, 7 aa, synthetic construct
SGASGAG

SEQ ID NO: 21 - protein, 10 aa, synthetic construct
DISVLMKEYQ

SEQ ID NO: 22 - protein, 10 aa, synthetic construct
DISVLMKEYD

SEQ ID NO: 23 - protein, 10 aa, synthetic construct
DIDVLMKEYD

SEQ ID NO: 24 - protein, 10 aa, synthetic construct
DIHVLMKEYD

SEQ ID NO: 25 - protein, 10 aa, synthetic construct
DILVLMKEYD

SEQ ID NO: 26 - protein, 10 aa, synthetic construct
DIPVLMKEYD

SEQ ID NO: 27 - protein, 10 aa, synthetic construct
DITVLMKEYD

SEQ ID NO: 28 - protein, 10 aa, synthetic construct
DISLLMKEYD

SEQ ID NO: 29 - protein, 10 aa, synthetic construct
DISVIMKEYD

SEQ ID NO: 30 - protein, 10 aa, synthetic construct
DISVVMKEYD

SEQ ID NO: 31 - protein, 10 aa, synthetic construct
DISVLDKEYD

SEQ ID NO: 32 - protein, 10 aa, synthetic construct
DISVLNKEYD

SEQ ID NO: 33 - protein, 10 aa, synthetic construct
DISVLCKEYD

SEQ ID NO: 34 - protein, 10 aa, synthetic construct
DISVLKKEYD

SEQ ID NO: 35 - protein, 10 aa, synthetic construct
DISVLEKEYD

SEQ ID NO: 36 - protein, 10 aa, synthetic construct
DISVLQKEYD

SEQ ID NO: 37 - protein, 10 aa, synthetic construct
DISVLMKEYN

SEQ ID NO: 38 - protein, 10 aa, synthetic construct
DISVLMKEYE

SEQ ID NO: 39 - protein, 10 aa, synthetic construct
DISVLSKEYD

SEQ ID NO: 40 - protein, 10 aa, synthetic construct
DISVLMKDYD

SEQ ID NO: 41 - protein, 10 aa, synthetic construct
DISVLMKEFD

SEQ ID NO: 42 - protein, 10 aa, synthetic construct
DISVLMKESD

SEQ ID NO: 43 - protein, 10 aa, synthetic construct
DISVLMKETD

SEQ ID NO: 44 - protein, 10 aa, synthetic construct
NISVLMKEYD

SEQ ID NO: 45 - protein, 10 aa, synthetic construct
DISELMKEYD

SEQ ID NO: 46 - protein, 10 aa, synthetic construct
DISVLLKEYD

SEQ ID NO: 47 - protein, 10 aa, synthetic construct
DISVLMKHYD

SEQ ID NO: 48 - protein, 10 aa, synthetic construct
DITVLCKEYN

SEQ ID NO: 49 - protein, 10 aa, synthetic construct
DIHVLDKEYD

SEQ ID NO: 50 - protein, 22 aa, synthetic construct
CGTGAPPDISVLMKEYQPSGAC

SEQ ID NO: 51 - protein, 28 aa, synthetic construct
SGAGGTGAPPDISVLMKEYQPSGASGAG

SEQ ID NO: 52 - protein, 471 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKCGTGAPPDISVLMKEYQPSGACTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 477 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKSGAGGTGAPPDISVLMKEYQPSGASGAGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 242 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGAGGTGAPPDISVLMKEYQPSGASGAG